(19) United States
(12) Patent Application Publication
(10) Pub. No.: US 2024/0108792 A1
(43) Pub. Date: Apr. 4, 2024

(54) PARYLENE COATED NON-MEDICAL CONSUMER PRODUCTS

(71) Applicant: Voopray, Sparks, NV (US)

(72) Inventors: Malekos Steven, Sparks, NV (US); Malekos Tamara, Sparks, NV (US)

(21) Appl. No.: 17/957,149

(22) Filed: Sep. 30, 2022

(51) Int. Cl. A61L 31/10

(52) U.S. Cl. CPC A61L 31/10; A61L 2420/02

(57) ABSTRACT

Disclosed are non-medical consumer products including a substrate that is coated with a layer of parylene. Particularly, the non-medical consumer products can include novelty items, such as adult novelty items, and children's products, such as pacifiers or toys. The layer of parylene extends lifetime of the non-medical consumer products and prevents bacterial growth thereon whilst also providing a protective barrier for a user of the non-medical consumer products, preventing adverse effects thereto from bioincompatible materials and toxic chemicals. A method of coating such items with parylene is also disclosed.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of non-medical consumer products of existing art and more specifically relates to parylene coated non-medical consumer products.

### RELATED ART

Production of plastic is known to leave behind residual monomers, some of which have been classified as hazardous or toxic substances. Monomers with genotoxic carcinogenic properties can exist in plastic materials used in consumer products from children's toys to adult novelty items. This is of particular concern for consumer products that come into contact with mucous membranes of a human body, such as adult novelty items, teething rings, pacifiers, etc.

The adult novelty market in particular is an unregulated industry and many adult novelty items are manufactured with materials known to cause health hazards. Common plastic material used in the adult novelty market include Acrylonitrile Butadiene Styrene (ABS), Polyvinyl Chloride (PVC), Polystyrene (PS), Styrene-Ethylene-Butylene-Styrene (SEBS), Styrene Butadiene Block Copolymer (SBC), Rubber, Thermoplastic Elastomer (TPE), Thermoplastic Polyolefins (TPO), Vinyl, Latex, Cyberskin, Fanta Flesh, etc.

There are a range of toxic, harmful chemicals found in these materials. For example, DEHP (Bis-2-ethylhexyl phthalate), which can cause harmful effects to a fetus, fertility problems, and harmful effects on liver and kidneys; cyclohexanone, which can be an irritant to mucous membranes, includes a risk of contact dermatitis in sensitive individuals and is a confirmed animal carcinogen; 2-methylhexanoic acid, which can potentially cause harmful effects to a fetus; carbon disulfide, which can potentially cause harmful effects to a fetus and on fertility; trimethyltin chloride, which can cause irreversible neurotoxic effects; tetrahydrofuran which can be irritating to eyes, the respiratory system, skin and mucous membranes and is a confirmed animal carcinogen; and phenol which can cause minor risk of health effects.

In many cases, the risk can also be increased depending on use of the adult novelty item. For example, if incompatible substances are added to the adult novelty item, such as lubricants or cleaning agents, the adult novelty item can quickly begin to degrade, shedding more harmful particles. Further, many materials used in adult novelty items are porous, which make them difficult to clean and keep free from bacteria growth.

In contrast, parylene is a “green”, robust polymer known to be chemically inert. It is resistant to almost every solvent, acid and alkaline chemistry commonly used. The U.S. Food and Drug Administration has approved parylene with a Class VI biocompatibility rating suitable for human implantable devices, based on its performance in the USPO Class VI grade and ISO 10993 group standards. Parylene is also a dry lubricity material that has a low coefficient of friction—five times less friction than silicone and 3 times less friction than glass.

As such, there exists a need to utilize the beneficial properties of parylene on products that particularly contact the human body, and more specifically, mucous membranes of the human body, to prevent shedding of dangerous chemicals, prolong lifetime of products by preventing degradation, decrease amount of lubrication needed due to low friction, and provide a less porous and thus easier cleanable surface.

### SUMMARY OF THE INVENTION

In view of the foregoing disadvantages inherent in the known novelty item art, the present disclosure provides novel parylene coated non-medical consumer products. The general purpose of the present disclosure, which will be described subsequently in greater detail, is to coat non-medical consumer products, such as adult novelty items, with a transparent thin film of parylene. Particularly, the thin film of parylene encapsulates harmful chemicals common in non-medical consumer products, prevents degradations associated with the use of non-compatible lubricants or cleaning agents, provides chemical compatibility with all lubricants and cleaning agents, decreases amount of artificial lubrication needed due to low coefficient friction of the parylene, and provides a non-porous surface for the non-medical consumer products, thereby preventing bacterial growth, unpleasant odors and degradation and improving sanitation and lifetime of the non-medical consumer products.

A non-medical consumer product is disclosed herein. The non-medical consumer product is configured for use in contact with mucus membrane tissue of a human body. The non-medical consumer product includes a non-medical consumer product substrate and the non-medical consumer product substrate is coated with parylene. Examples of non-medical consumer products may include, but are not limited to, pelvic floor dilators, vaginal dilators, pacifiers, nasal cannulas, etc.

An adult novelty item is disclosed herein. In some embodiments, the adult novelty item may also be particularly configured for contact with the human body, or more specifically but not limited to, a mucus membrane of the human body. The adult novelty item includes an adult novelty substrate and the adult novelty substrate is coated with parylene. In some examples, the adult novelty item may include, but is not limited to, dildos, vibrators, clitoral vibrators, male masturbators, penis rings, penis pumps, clitoral pumps, sex dolls, oral sex toys, penis sleeves, penis extensions, anal toys, butt plugs, strap-ons, nipple toys, or the like.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, parylene coated non-medical consumer products, constructed and operative according to the teachings of the present disclosure.

FIG. 1 is a cross-sectional view of a non-medical consumer product having a non-medical consumer product substrate coated with a layer of parylene, according to an embodiment of the disclosure.

FIG. 2 is a partial magnified view of the non-medical consumer product wherein the non-medical consumer product is a pacifier coated with the layer of parylene, according to an embodiment of the disclosure.

FIG. 3 is a cross-sectional view of an adult novelty item having an adult novelty substrate coated with the layer of parylene, according to another embodiment of the disclosure.

FIG. 4 is a partial magnified view of the adult novelty item wherein the adult novelty is a sex toy coated with the layer of parylene, according to another embodiment of the disclosure.

FIG. 5 is a pictorial diagram illustrating a process and apparatus for coating a substrate with a layer of parylene, according to another embodiment of the disclosure.

FIG. 6 is a front view a non-medical consumer product including a tether, according to an embodiment of the disclosure.

FIG. 7 is a perspective view a plurality of non-medical consumer products being hung from a fixture via their respective tethers, according to an embodiment of the disclosure.

FIG. 8 is a bar graph illustrating wear index of parylene N and parylene C as compared with other materials, according to another embodiment of the disclosure.

FIG. 9 is a bar graph illustrating coefficient of friction of parylene N, parylene C, and parylene F as compared with other materials, according to another embodiment of the disclosure.

FIG. 10 is a bar graph illustrating moisture vapor transmission of parylene N, parylene C, and parylene F as compared with other materials, according to another embodiment of the disclosure.

FIG. 11 is a table illustrating gas permeability of parylene N, parylene C, and parylene F, according to another embodiment of the disclosure.

The various embodiments of the present invention will hereinafter be described in conjunction with the appended drawings, wherein like designations denote like elements.

### DETAILED DESCRIPTION

As discussed above, embodiments of the present disclosure relate to non-medical consumer products and more particularly to parylene coated non-medical consumer products. Generally, the present disclosure may include novelty items, such as adult novelty items or children's toys, coated with parylene. Encapsulating these products with a parylene coating aids in chemical compatibility with common cleaning agents and lubricants whilst keeping preferred texture or feel of product materials, increases lubrication, and provides a non-porous and thus easily cleanable surface.

Further, encapsulating the products with the parylene coating provides a diffusion barrier to help conceal harmful chemicals and prevent potential adverse side effects commonly associated with porosity (bacteria growth), phthalates, toxic chemicals, rubber, thermoplastic epoxy, thermoplastic resin, vinyl, polyvinyl chloride, acrylonitrile butadiene styrene, silicone, latex, parabens, thermo elastic material, etc.

In some embodiments, a traceability system may be implemented in order to keep track of all parylene coated non-medical consumer products. For example, a user may be provided with a card having a barcode, such as (but not limited to) a QR code. The QR code, when scanned, may provide access to a database which shows specific account information associated with the card. This may aid in product recalls. Further, the barcode may also be used for validation of authenticity.

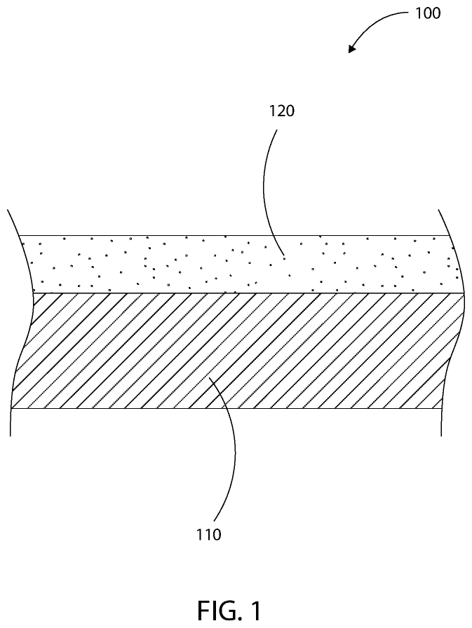

Referring now more specifically to the drawings by numerals of reference, there is shown in FIGS. 1-11, various views of a non-medical consumer product 100 and an adult novelty item 200. Particularly, the non-medical consumer product 100 may be configured for use in contact with mucus membrane tissue of a human body. For example, the non-medical consumer product 100 may include, but is not limited to, a pelvic floor dilator, a vaginal dilator, a pacifier, an adult novelty item, nasal cannulas, etc.

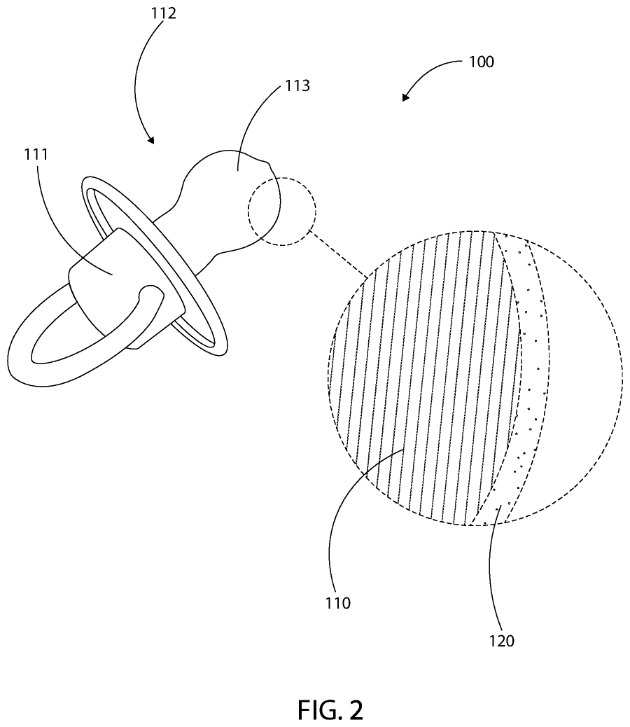

As shown particularly in FIGS. 1-2, the non-medical consumer product 100 may include a non-medical consumer product substrate 110 coated with parylene 120. For example, as shown, a layer of parylene 120 may be applied over the non-medical consumer product substrate 110. In some embodiments, the layer of parylene 120 may be applied to the non-medical consumer product substrate 110 at a location thereon designed for contact with the mucus membrane of the human body, thereby preventing chemical leaching into the human body, outgas sing of undesirable Volatile Organic Compounds (VOC's), chemical burns, allergic reactions, carcinogen exposure, etc.

For example, as above and as demonstrated in FIG. 2, the non-medical consumer product 100 may include a pacifier 112. Particularly, the pacifier 112 may include a handle element 111 and a bulb element 113 (the bulb element 113 naturally being the portion in which contacts the mouth (mucus membrane) of an infant). As such, the layer of parylene 120 may be specifically applied to the bulb element 113, as shown in FIG. 2. However, it should be appreciated that the layer of parylene 120 is not limited to the bulb element 113, and the handle element 111 may also be coated with the layer of parylene 120.

In some embodiments, the non-medical consumer product 100 may include one or more silicone components. In these embodiments, the layer of parylene 120 may be applied to the silicone components to allow the non-medical consumer product 100 to be compatible with silicone lubricant. This may be particularly useful for items such as (but not limited to) pelvic floor dilators, vaginal dilators, and adult novelty items, which typically require the use of silicone lubricant. This may prevent degradation of the non-medical consumer product 100, extend lifetime of the medical consumer product, provide an easily cleanable surface, etc.

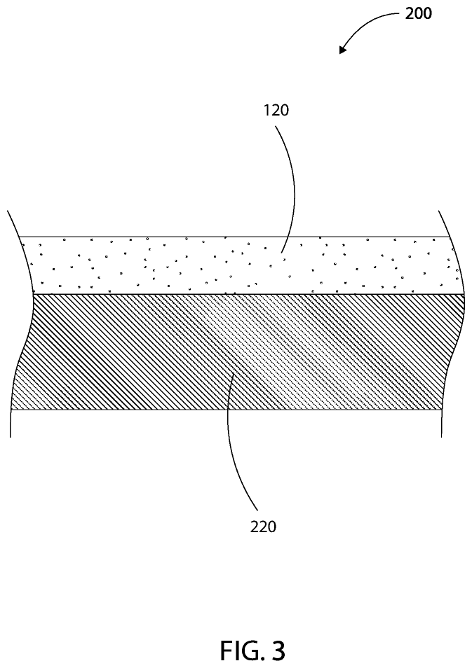

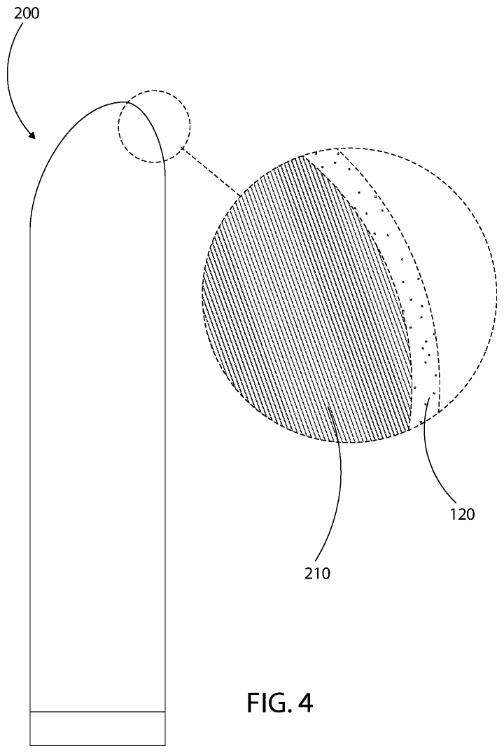

In another embodiment of the present disclosure, as demonstrated in FIGS. 3-4 particularly, an adult novelty item 200 (or more specifically, a sex toy) may be disclosed. Some examples of adult novelty items 200 contemplated may include, but are not limited to, a dildo, a vibrator, a clitoral vibrator, a male masturbator, a penis ring, a penis pump, a clitoral pump, a sex doll, an oral sex toy, a penis sleeve, a penis extension, an anal toy, a butt plug, a strap-on, a nipple toy, etc.

The adult novelty item 200 may include an adult novelty substrate 210 coated with parylene 120. Again, similar to the non-medical consumer product 100 discussed above, the layer of parylene 120 may be applied over the adult novelty substrate 210. The layer of parylene 120 may be applied to the adult novelty substrate 210 at a location on the adult novelty substrate 210 that is designed for contact with a human body. Particularly, the layer of parylene 120 may be applied to the adult novelty substrate 210 at a location on the adult novelty substrate 210 that is designed for contact with a mucus membrane of the human body.

Further, the adult novelty item 200 may include one or more silicone components and the layer of parylene 120 may particularly allow the adult novelty item 200 to be compatible with silicone lubricant. Again, this may prevent degradation of the adult novelty item 200, extend lifetime of the non-medical consumer product, provide an easily cleanable surface, etc.

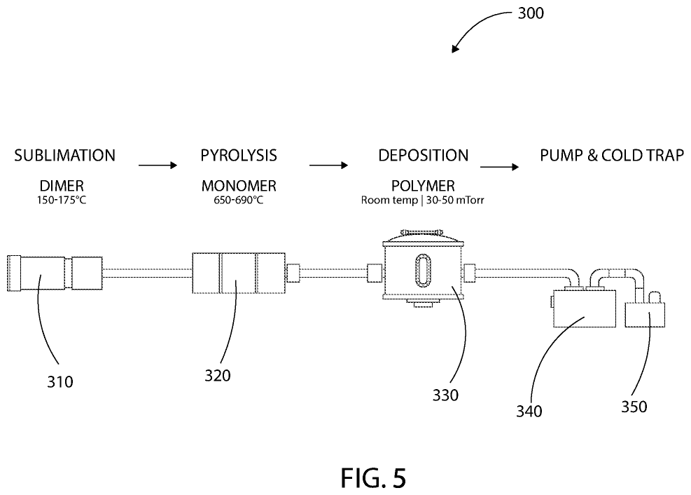

Referring now more specifically to FIG. 5 illustrating a parylene coating process 300 with apparatus used in the process. Parylene films are “grown” as vapor deposits molecule by molecule in a room temperature vacuum chamber. This process provides unique benefits over dip and spray coatings; namely, the vapor is able to conform to all ‘nooks and crannies’ of a substrate (anywhere the vapor reaches). As such, the result is a pinhole free, transparent, equally and uniformly conforming coating on all exposed surfaces of products being coated. Accordingly, the layer of parylene 120 may be a thin film which is highly uniform, ranging from hundreds of angstroms to hundreds of microns. The layer of parylene 120 protects the substrate, whether it be a simple structure or complex, at a molecular level; encapsulating the substrate and evenly covering sharp edges.

As shown in FIG. 5, the parylene coating process may begin with “sublimation”. Particularly, granular dimer may be vaporized within a vaporizer 310 at 150-175 degrees Celsius, turning the granular dimer from a solid into a dimeric gas. Next, “pyrolysis” may include the dimeric gas being heated at 650-690 degrees Celsius in a pyrolysis furnace 320, splitting the dimer molecule and producing a monomeric gas. Next, a “deposition” step includes coating the substrate in a coating chamber 330, whereby monomer gas is released into the coating chamber 330 to deposit onto the substrate as a transparent, uniformly conforming polymer film. This step is achieved at room temperature and 30-50 mTorr. A vacuum pump 340 is provided and configured to remove all air and gasses from the coating chamber 330 to enable parylene deposition. The parylene will penetrate beneath, between, inside, around and into microscopic devices; there may be no air gaps, voids or edge effects. Finally, a cold trap device 350 captures excess parylene at the end of the deposition step.

Using Parylene C, the deposition rate may be approximately 5 um per hour (0.0002″ per hour); and for Parylene N, the deposition rate may be 762 nm per hour (0.00003″ per hour). The deposition rate may be indirectly controlled by regulating pressure in the coating chamber 330.

The coating chamber 330 may preferably include a large viewport, allowing for full view of load and easy continuous monitoring thereof; an adjustable, single port baffle for easy maintenance; separate pressure calibration/verification port for easy and repeatable verification; a vapor silane port; an auxiliary port to add an ability to integrate optional horizontal tumbler or other feedthrough tools, which may be 2.5 inches in diameter; and variable speed, direct drive fixture/product rotation. Interior dimensions of the coating chamber 330 may be 24 inches in diameter and 28 inches in depth and may be built to accommodate product weight loads of up to 300 pounds. Further, the coating chamber 330 may be made from 304 stainless steel material and be electropolished on the interior and exterior. It should however be appreciated that the coating chamber 330 is not limited to any of these features.

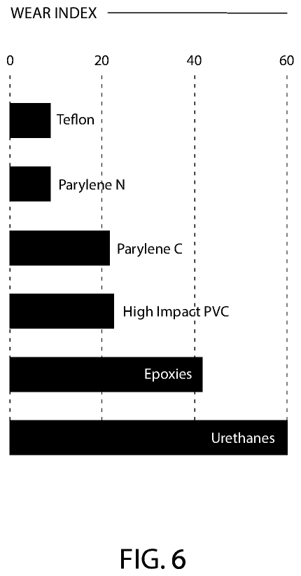

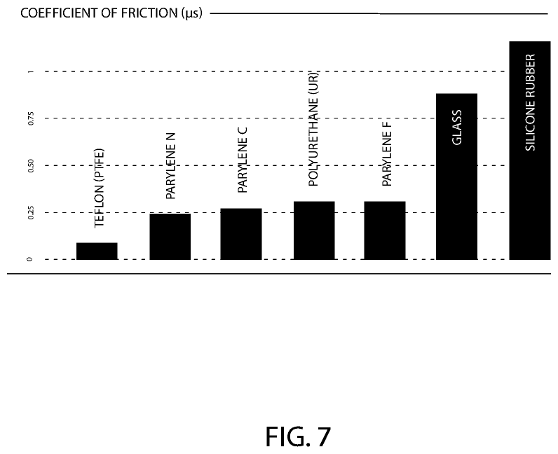

Referring more specifically to FIGS. 6-7, there is shown the non-medical consumer product 100 including a tether 114. The tether 114 may be used to attach the non-medical consumer product 100 to a fixture 130 (as shown in FIG. 7). Particularly, as shown in FIG. 6, the tether 114 may include a length of material 115 and a ball 116 attached to an end of the length of material 115. As shown in FIG. 7, the fixture 130 may include a plurality of tether holders 131 each being configured to grasp the ball 116 of the tether 114 therein. As such, the non-medical consumer product 100 is able to be hung from the fixture 130 during the coating process. This maximizes the surface area able to be coated with the layer of parylene 120 and increases the number of non-medical consumer products 100 that are able to be coated in a single deposition (as demonstrated in FIG. 7). After the coating process, the tether 114 may be removed from the non-medical consumer product 100. For example, this may be accomplished during a post processing manufacturing step.

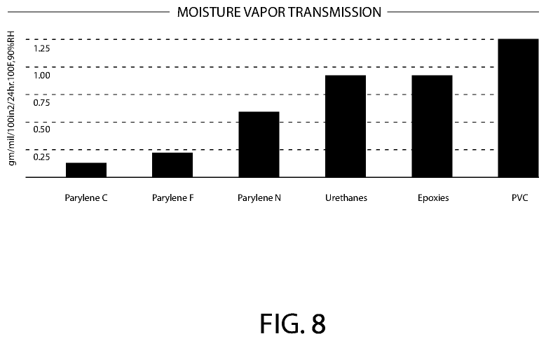

In all embodiments, type of parylene (whether parylene C, parylene N or parylene F, or other) may be chosen based on the type of substrate. FIGS. 8-11 illustrate properties of parylene; in particular, barrier properties, gas permeability, mechanical properties and wear index of parylene C, parylene N or parylene F. As discussed above, coating the substrate, or product, with the layer of parylene 120 aids in chemical compatibility with common cleaning agents and lubricants, acts as a diffusion barrier to help conceal harmful chemicals, increases lubrication and provides a non-porous and easily cleanable surface.

Particularly, parylene is insoluble to all common solvents, acids and alkalis. As such, the layer of parylene 120 enables the product/substrate to be thoroughly cleaned with any cleaning agent and used with any lubrication, including, but not limited to, water-based lubricants, silicone-based lubricants, organic lubricants, flavored lubricants, hybrid lubricants, warming lubricants, petroleum-based lubricants, etc. As such, the lifetime of the product may be substantially improved. FIG. 8 demonstrates the wear index of parylene N and parylene C compared with Teflon, High Impact PVC, epoxies and urethanes. It is contemplated that the layer of parylene 120 may provide relatively high tensile and yield strength without causing adverse reactions for a user of the product, as the parylene also includes a hardness approximately equal to human skin.

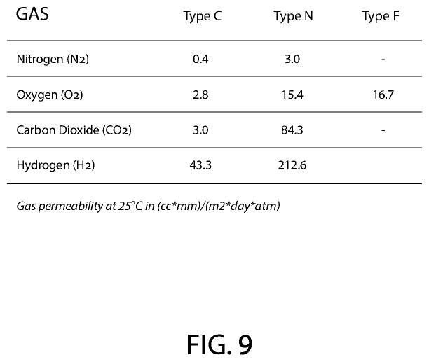

As demonstrated via the graph in FIG. 9, parylene is also a dry lubricity material with a low coefficient of friction, and as such, the layer of parylene 120 on the product, particularly adult novelty items 200 as discussed above, may decrease amount of artificial lubrication needed. Further, as demonstrated via the graphs in FIGS. 10-11, the layer of parylene 120 acts as an extremely effective moisture and chemical barrier; and as such, when applied to the substrate, it encapsulates harmful and toxic chemicals present in the substrate/product material and prevents the harmful and toxic chemicals shedding onto or into a user of the product, therefore preventing adverse reactions or side effects to the user.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

The numbered list format used in the claims of the application is used solely for organizational purposes to provide clarity thereto and is not meant to limit in any way the claimed matter nor any aspect of the present disclosure.

What is claimed is:

1. A non-medical consumer product for use in contact with mucus membrane tissue of a human body comprising:
a. a non-medical consumer product substrate;
b. wherein the non-medical consumer product substrate is coated with parylene.

2. The non-medical consumer product of claim 1, wherein the non-medical consumer product substrate includes one or more silicone components, wherein a layer of parylene is applied to the silicone components thereby allowing the non-medical consumer product to be compatible with silicone lubricant.

3. The non-medical consumer product of claim 1, wherein the non-medical consumer product substrate comprises a pelvic floor dilator.

4. The non-medical consumer product of claim 1, wherein the non-medical consumer product substrate comprises a vaginal dilator.

5. The non-medical consumer product of claim 1, wherein a layer of parylene is applied to the non-medical consumer product substrate at a location on non-medical consumer product substrate that is designed for contact with a mucus membrane of a human body.

6. The non-medical consumer product of claim 5, wherein the non-medical consumer product comprises a pacifier, the pacifier having a handle element and a bulb element, wherein the layer of parylene is applied to the bulb element.

7. An adult novelty item comprising:
a. an adult novelty substrate;
b. wherein the adult novelty substrate is coated with parylene.

8. The adult novelty item of claim 7, wherein the adult novelty substrate is selected from the group consisting of a dildo, a vibrator, a clitoral vibrator, a male masturbator, a penis ring, a penis pump, a clitoral pump, a sex doll, an oral sex toy, a penis sleeve, a penis extension, an anal toy, a butt plug, a strap-on, and a nipple toy.

9. The adult novelty item of claim 7, wherein a layer of parylene is applied to the adult novelty substrate at a location on the adult novelty substrate that is designed for contact with a human body.

10. The adult novelty item of claim 7, wherein a layer of parylene is applied to the adult novelty substrate at a location on the adult novelty substrate that is designed for contact with a mucus membrane of a human body.

11. The adult novelty item of claim 7, the adult novelty item including one or more silicone components, wherein a layer of parylene is applied to the silicone components thereby allowing the adult novelty item to be compatible with silicone lubricant.

12. A process for coating a substrate with parylene comprising the steps of:
a. sublimating a granular parylene dimer from a solid phase to a dimeric gas by heating to a temperature range of 150 to 175 degrees centigrade;
b. converting the dimeric gas to a monomeric case through pyrolysis by heating to a temperature range of 650 to 690 degrees centigrade;
c. placing the substrate in a room-temperature coating vacuum chamber, the pressure inside the room-temperature coating vacuum chamber is in the range of 30 to 50 mTorr;
d. releasing the monomeric gas into the room-temperature coating vacuum chamber whereby the monomeric gas is deposited upon the substrate as a transparent, uniformly conforming polymer film of parylene.

13. The method of claim 12, further comprising the step of capturing excess parylene at the end of the deposition step using a cold trap device.

14. The method of claim 12, wherein the substrate comprises a non-medical consumer product.

15. The method of claim 14, wherein during the deposition step, the substrate is attached to a tether and the tether is hung from a fixture inside the room-temperature coating vacuum chamber thereby maximizing an amount of surface area of the substrate coated and thereby increasing the number of substrates that may be coated at once.

16. The method of claim 15, wherein the tether comprises a ball attached to an end of a length of material.

17. The method of claim 16, wherein the fixture includes a plurality of tether holders each being configured to grasp the ball of the tether.
